(19) United States
(12) Patent Application Publication
(10) Pub. No.: US 2024/0115579 A1
(43) Pub. Date: Apr. 11, 2024

(54) PHARMACEUTICAL COMPOSITIONS AND MANUFACTURING METHODS THEREOF

(71) Applicant: Galenicum Health S.L.U., Esplugues de Llobregat (ES)

(72) Inventors: Luis Gómez Coello, San Agustín del Guadalix (ES); Maria del Rocío Pérez Pérez, San Agustín del Guadalix (ES)

(73) Assignee: Galenicum Health S.L.U., Esplugues de Llobregat (ES)

(21) Appl. No.: 18/470,208

(22) Filed: Sep. 19, 2023

(30) Foreign Application Priority Data
Sep. 22, 2022 (EP) 22382874.0
Apr. 28, 2023 (EP) 23382405.1

(51) Int. Cl. A61K 31/5415; A61K 33/06; A61K 33/26

(52) U.S. Cl. CPC A61K 31/5415; A61K 33/06; A61K 33/26

(57) ABSTRACT

A sterile aqueous pharmaceutical composition contains methylene blue as active ingredient and water, wherein an Azure B impurity content of the composition ranges from 2.0 wt. % to 3.0 wt. %, a pH of the composition is below 4.0, and a total metal content of the pharmaceutical composition is above 20 ppm.

### CROSS-REFERENCE TO RELATED APPLICATIONS

This application claims priority to European Application No. EP22382874.0, filed on Sep. 22, 2022, and European Application No. EP23382405.1, filed on Apr. 28, 2023, the content of each of which is hereby incorporated by reference in its entirety.

### FIELD

The present invention relates to a pharmaceutical composition comprising methylene blue. Manufacturing processes of such compositions are also described.

### BACKGROUND

Methylthioninium chloride, also known as methylene blue or, 3,7-bis-(dimethylamino)-phenothiazin-5-ium chloride, is an oxidation-reduction agent used for the treatment of patients with acquired methemoglobinemia.

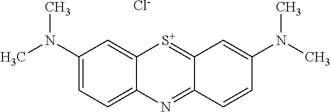

Methylene blue is an old known compound. According to Merck Index, 8th Edition (1964), it was first described in a German Patent in 1877 (Badische Anilin und Soda Fabrik, 1877).

Methylene Blue compounds used in the present invention can be obtained according to the processes disclosed in WO 2018/167185.

WO 2006/032879 and WO 2008/007074 disclose a high purity diaminophenothiazonium compound with a purity greater than 98%, an Azure B impurity content of less than 2% and a low content of certain metals. Moreover, this document discloses that in the different steps of the process, care is taken to use non-metallic materials, reagents and solvents free of metal residues and that the purification and hydration steps are performed in an enamelled reactor under nitrogen.

WO 2008/006979 discloses a method for preparing methylene blue with a low level of impurities and low metal content.

The European Public Assessment report for Methylthioninium chloride Proveblue discloses that methylene blue has a very strong affinity for metals and that it is difficult to manufacture a substance that complies with the requirements of Ph. Eur. Moreover, it discloses that methylene blue even absorbs metals from the brown vials where it is stored.

US 2022/0265674 discloses pharmaceutical composition of methylene blue comprising a pH adjuster. The incorporation of a pH adjuster in a pharmaceutical composition has certain drawbacks. Firstly, it can lead to undesirable chemical reactions that can negatively impact the efficacy and stability of the active ingredient. Additionally, the presence of a pH adjuster can increase the likelihood of microbial growth, which can compromise the safety of the product, something of great concern in a parenterally administered product. Moreover, adding manufacturing steps (and/or excipients) increases the cost of manufacturing a pharmaceutical product. Therefore, careful consideration should be given to the use of pH adjusters in pharmaceutical compositions to minimise potential disadvantages and ensure optimal performance and safety of the medication. The inventors have found out that avoiding traditional pH adjusters in a methylene blue composition has substantial benefits.

There is a need in the field of pharmaceutical manufacturing for a stable, safe and effective pharmaceutical composition of methylene blue wherein said methylene blue contains a higher level of impurities than the prior art.

### SUMMARY OF THE INVENTION

The present invention discloses pharmaceutical compositions comprising methylene blue and methods for preparing said compositions, wherein the active substance has significantly higher impurity levels and metal content than those identified in the prior art. The inventors have surprisingly found that a very specific pH is required in said pharmaceutical compositions in order to obtain a stable pharmaceutical composition.

The first aspect of the invention deals with a sterile aqueous pharmaceutical composition consisting of methylene blue and water, wherein said methylene blue has an Azure B impurity content ranging between 2% and 3% and wherein the pH of said composition is below 4.0.

A second aspect of the invention deals with a process for the preparation of said pharmaceutical composition.

A third aspect of the invention deals with an industrial pharmaceutical batch of the first aspect using the process of the second aspect of the invention.

The invention includes but is not limited to the following embodiments:
1. A sterile aqueous pharmaceutical composition, consisting of: methylene blue as active ingredient and water, wherein
   a. an Azure B impurity content of said composition ranges from 2.0 wt. % to 3.0 wt. %, with respect to the active ingredient,
   b. a pH of said composition is below 4.0, and
   c. a total metal content of said pharmaceutical composition is above 20 ppm.
2. The pharmaceutical composition according to embodiment 1, wherein the Azure B impurity content of said composition ranges from 2.1 wt. % to 3.0 wt. %.
3. The pharmaceutical composition according to embodiment 1, wherein the pH is from 3.2 to 3.9.
4. The pharmaceutical composition according to embodiment 1, wherein the metal content in the composition is above 120 ppm.
5. The pharmaceutical composition according to embodiment 1, wherein an Iron (Fe) content with respect to the active ingredient is above 20 ppm.
6. The pharmaceutical composition according to embodiment 1, wherein an Iron (Fe) content with respect to the active ingredient is above 100 ppm.
7. The pharmaceutical composition according to embodiment 1, wherein an aluminum (Al) content with respect to the active ingredient is above 5 ppm.
8. The pharmaceutical composition according to embodiment 1, wherein the pharmaceutical composition is packaged in a glass vial, or in a pre-filled-syringe, or in a cartridge, or in an ampule.
9. The pharmaceutical composition according to embodiment 1, wherein the composition is suitable to be parenterally administered to a human.
10. A medicament, comprising: the pharmaceutical composition according to embodiment 1.
11. A method of treating dementia, cognitive impairment, Alzheimer's disease or methemoglobinemia, said method comprising: administering the pharmaceutical composition according to embodiment 1 to a subject in need thereof.
12. The pharmaceutical composition according to embodiment 1, having an assay value within 95 wt. %-105 wt. % and a total organic impurity content below 3 wt. % after 6 months storage under 40° C./75% RH (relative humidity) packaged in a glass vial with a rubber stopper.
13. The pharmaceutical composition according to embodiment 1, wherein said composition is an aqueous solution and a methylene blue concentration in the aqueous solution is 5 mg/mL.
14. A process for preparing a pharmaceutical composition according to embodiment 1, said process comprising:
   a. weighing the methylene blue,
   b. in a reactor, dissolving and mixing the methylene blue of step a. in water to obtain an aqueous solution,
   c. optionally, filtering the obtained aqueous solution,
   d. filling the aqueous solution into a suitable pharmaceutical container,
   e. sterilizing the obtained solution, thereby obtaining a sterile aqueous solution of said pharmaceutical composition.
15. The process according to embodiment 14, wherein the reactor material is metallic.
16. The process according to embodiment 14, wherein the solution in step c) is filtered through a 0.2 μm pore size filter.
17. The process according to embodiment 14, wherein a filtration through a 0.2 μm pore size filter is performed twice.
18. The process according to embodiment 14, wherein the suitable pharmaceutical container is a glass vial sealed with a rubber stopper.
19. The process according to embodiment 14, wherein a pH of the aqueous solution of step d ranges between 3.2 and 3.9.
20. The process according to embodiment 14, wherein at least 15 liters of sterile aqueous solution are obtained.
21. A sterile aqueous pharmaceutical composition, comprising methylene blue as active ingredient, at least one metal, Azure B impurity and water, wherein
   a. the Azure B impurity content of said composition ranges from 2.0 wt. % to 3.0 wt. %, with respect to the active ingredient,
   b. a pH of said composition is below 4.0, and
   c. a total metal content of said pharmaceutical composition is above 20 ppm, and wherein said composition is free from pH adjusters.
22. A sterile aqueous pharmaceutical composition, comprising methylene blue as active ingredient at a concentration of 5 mg/mL, at least one metal, Azure B impurity and water, wherein
   d. the Azure B impurity content of said composition ranges from 2.0 wt. % to 3.0 wt. %, with respect to the active ingredient,
   e. a pH of said composition is below 4.0, and
   f. a total metal content of said pharmaceutical composition is above 20 ppm, and wherein said composition is free from pH adjusters.

### DETAILED DESCRIPTION OF THE INVENTION

### Definitions

As used in the present disclosure, the following words, phrases, and symbols are generally intended to have the meanings as set forth below, except to the extent that the context in which they are used indicates otherwise.

All percentages are expressed by weight (w/w) specifically noted otherwise.

The term “active ingredient” as used herein refers to a therapeutically active compound, as well as any pharmaceutically acceptable hydrates and solvates of the compound.

In one embodiment, the term “consisting of” as used herein refers as to that no further features are present in the apparatus/method/product apart from the ones following said wording. It is considered that the methylene blue impurities such as Azure B are inherent to any methylene blue compound, as well as any other impurity (including metal impurities) that the API carries. Therefore, in the present application, the term “consisting of methylene blue as active ingredient and water” encompasses all methylene blue impurities.

The term “methylene blue impurities” include but are not limited to Azure A, Azure B, Azure C and any unspecified degradation product from methylene blue or its impurities.

The term “pH adjuster” as used herein refers to pharmaceutically acceptable excipients which are added to the solution of the active agent to adjust the pH to a certain value. Such pH adjusters can be alkaline or acid agents and may comprise inorganic salts as well as organic acids or salts of organic acids. Additionally, the pH adjusters may be present in the form of a buffer.

The term “pharmaceutically acceptable” as used herein indicates that the substance or composition must be compatible chemically and/or toxicologically, with the other ingredients comprising a formulation, and/or the mammal being treated therewith.

The term “effective” amount as used herein refers to an amount of a compound, agent or substance that is of sufficient quantity to result in a quantifiable effect, e.g., a shift in the pH.

The term “solution” as used herein refers to a liquid preparation of one or more soluble chemical substances, which are dissolved in water.

The term “stable” as used herein refers to any parenteral solution of methylene blue as active ingredient having a sufficient physical and chemical stability to allow storage under any of the general storage conditions as defined by ICH Q1A (R2).

The term “suitable for parenteral administration” as used herein refers to a sterile solution that can be administered parenterally to a patient. Sterility is defined as the absence of living organisms. The conditions of the sterility test are given in the European Pharmacopoeia 10th edition.

The term “suitable pharmaceutical container” as used herein refers to a container for pharmaceutical use is an article which holds or is intended to contain and protect a drug and is or may be in direct contact with it. The container and its closure must not interact physically or chemically with the substance within in any way that would alter its quality.

The term “vial” as used herein refers to a vial suitable for containing injectable pharmaceutical compositions according to the European Pharmacopoeia 9th edition, Chapter 3.2.1 “Glass Containers for Pharmaceutical Use”.

The term “terminal sterilization” as used herein refers to a process whereby a product is sterilized in its final container or packaging, which permits the measurement and evaluation of quantifiable microbial lethality. The probability of viable microorganisms being present on a product unit after exposure to the proper sterilization process should be less than 10⁻⁶.

The term “bioburden” as used herein refers to the population of viable microorganisms on or in raw materials, products, or labeling/packaging materials determined before sterilization. Bioburden analysis is carried out according to US Pharmacopoeia <71> Sterility tests.

The term “blanketing” as used herein refers to the act of maintaining an inert atmosphere of nitrogen gas (N₂) during storage and processing.

The term “0.2 μm pore filter”, as used herein refer to filter where the nominal pore rating have been determined to be 0.2 μm or less, according to ISO 13408-2:2018 Aseptic processing of health care products.

Unless otherwise indicated, all the analysis methods are carried out according to the European Pharmacopoeia 10th edition (monograph on page 3250 and 3251, 2020) and USP monograph for Methylene Blue Injection (USP43-NF38-2882).

The terms “Azure B impurity” or “Azure B” as used herein refer to 3-(dimethylamino)-7-(methylamino)phenothiazin-5-ylium. Percentage of Azure B is determined by HPLC (Column XBridge Phenyl 100, flow rate 1.0 mL/min, UV detector wavelength of 246 nm, column temperature 30° C., mobile phase: 0.1% trifluoroacetic acid in water:acetonitrile (75:25% v/v), run time 15 minutes).

The term “Azure B impurity content” refers to the percentage amount of Azure B in the pharmaceutical composition with respect to the active ingredient (w/w).

The term “total impurity content” refers to the percentage amount of the sum of all the impurities in the pharmaceutical composition with respect to the active ingredient (w/w).

The terms “purity” or “chemical purity” referred to herein refers to the measure of an element containing a single substance without any other element tarnishing its standalone existence. In the present invention, the purity calculations have been provided based on the total organic impurity content and the metal content.

The terms “metal purity” or “metal content” referred to herein pertains to the amounts of the eleven (11) metals specified by the European Pharmacopoeia: Al, Cr, Zn, Cu, Fe, Mn, Ni, Mo, Cd, Sn, and Pb. The metal content is expressed relative to the amount of the active ingredient. The metal content has been determined following Ph. Eur. 10th Edition, chapter 2.4.20—Determination of elemental impurities.

The inventors have found out that a sterile aqueous pharmaceutical composition comprising methylene blue as active ingredient wherein the active ingredient used to manufacture has not a high level of purity (total impurity content) as the methylene blue obtained as per the prior art can be surprisingly stable when the pH of said composition is below 4.0.

In a first aspect of the invention, a sterile aqueous pharmaceutical composition comprising methylene blue as active ingredient wherein the Azure B impurity content of said composition ranges from 2.0% to 3.0% and the pH of said composition is below 4.0.

In an embodiment of the first aspect of the invention, said aqueous pharmaceutical composition comprises an Azure B impurity content ranging from 2.1% to 3.0%, preferably from 2.3% to 3.0%.

In another embodiment, the Azure B impurity content can be taken as representative of the total impurity content of the pharmaceutical composition.

In another embodiment, the pH of said pharmaceutical composition ranges from 3.2 to 3.9, preferably from 3.3 to 3.8.

In another embodiment, the pharmaceutical composition of the invention is free from pH adjusters.

In another embodiment, the pharmaceutical composition of the invention is free from organic solvents. In a preferred embodiment, the solvent consists of water, more preferably, sterile water for injection.

In another embodiment, the said pharmaceutical composition has a concentration of 5 mg/mL of methylene blue or any of its hydrates.

In another embodiment, the metal content relative to the amount of methylene blue is above 20 ppm, preferably above 50 ppm, more preferably above 70 ppm, even more preferably above 100 ppm. In a further preferred embodiment, the content is above 120 ppm or above 130 ppm or above 150 ppm.

In another embodiment, the iron (Fe) content relative to the amount of methylene blue is above 20 ppm, preferably above 50 ppm, more preferably above 100 ppm, even more preferably above 120 ppm.

In another embodiment, said pharmaceutical composition is packaged in a glass vial sealed with a rubber stopper, or in a pre-filled-syringe, or in a cartridge or in a glass ampule, preferably the composition is packaged in a glass vial sealed with a rubber stopper.

In another embodiment, said pharmaceutical composition is to be parenterally administered to a human.

In another embodiment, said pharmaceutical composition is to be used as a medicament.

In another embodiment, said pharmaceutical composition is to be used in the treatment of dementia, cognitive impairment, Alzheimer or methemoglobinemia, preferably for the treatment of methemoglobinemia.

In another embodiment, the compositions show assay values within 95%-105% and total organic impurity content below 3% after 6 months storage under 40° C./75% RH (relative humidity) packaged in glass vials with rubber stopper.

In another embodiment, a sterile aqueous pharmaceutical composition comprising methylene blue as active ingredient, water, Azure B impurity and at least one metal, wherein the Azure B impurity content of said composition ranges from 2.0% to 3.0% with respect to the active ingredient, wherein the total metal content of said pharmaceutical composition is above 20 ppm, wherein the pH of said composition is below 4.0 and wherein the composition is free from pH adjusters is disclosed.

In another embodiment, a sterile aqueous pharmaceutical composition comprising methylene blue as active ingredient at a concentration of 5 mg/mL, water, Azure B impurity and at least one metal, wherein the Azure B impurity content of said composition ranges from 2.0% to 3.0% with respect to the active ingredient, wherein the total metal content of said pharmaceutical composition is above 20 ppm, wherein the pH of said composition is below 4.0 and wherein the composition is free from pH adjusters is disclosed.

In another embodiment, a sterile aqueous pharmaceutical composition consisting of methylene blue as active ingredient at a concentration of 5 mg/mL, water, methylene blue impurities and at least one metal, wherein the Azure B impurity content of said composition ranges from 2.0% to 3.0%, wherein the total metal content of said pharmaceutical composition is above 20 ppm, and wherein the pH of said composition is below 4.0 is disclosed.

In another embodiment, the composition further comprises not more than 0.20% of Azure A and not more than 0.20% of Azure C.

All the embodiments of the first aspect of the invention may be combined between them.

In a second aspect of the invention, a process for preparing said pharmaceutical composition is disclosed.

In an embodiment of the second aspect of the invention, a process for preparing the pharmaceutical composition of the first aspect of the invention comprising the steps of:
a. weighing the methylene blue,
b. in a reactor, dissolving and mixing methylene blue of step a. in water to obtain a solution
c. optionally, filtering the obtained solution,
d. filling the solution into a suitable pharmaceutical container,
e. sterilising the obtained solution

In a further embodiment, the methylene blue in step a. has a purity below 98% and an Azure B content of at least 2%.

In a further embodiment, the reactor of step b. is a reactor made of metal, preferably made of stainless steel.

In a further embodiment, the filtration of step c. is filtered through a 0.2 μm pore size filter.

In a further embodiment, the filtration of step c. is performed twice.

In a further embodiment, the bioburden of the solution before any filtration is higher than 10 CFU/100 mL. In case the bioburden before filtration is not more than 10 CFU/100 mL. the filtration stages may be waived.

In a further embodiment, step e. is performed by terminal sterilisation and preferably reaching at least 110° C. for not less than 10 minutes, more preferably reaching at least 120° C. for not less than 10 minutes.

In a further embodiment, the terminal sterilisation of step e. is performed in an autoclave.

In a further embodiment, the aqueous solution reaches a temperature inside the reactor of at least 20° C.

In a further embodiment, the suitable pharmaceutical container of step e. is a glass vial sealed with a rubber stopper.

In a further embodiment, the vial is blanketed with nitrogen before sealing.

In a further embodiment, the sterilisation process reaches at least 121° C. for at least 10 minutes, preferably 20 minutes.

In a further embodiment the sterilisation process is maintained between 121° C. and 124° C. in an autoclave during 20 minutes.

The embodiments of the second aspect of the invention may be combined between them to obtain the composition according to the first aspect of the invention. A preferred combination of embodiments is as follows:

A process for preparing a sterile aqueous pharmaceutical composition comprising methylene blue as active ingredient wherein the Azure B impurity content of said composition ranges from 2.0% to 3.0% and the pH of said composition is below 4.0 comprising the steps of:
a. weighing the methylene blue having a purity below 98% and an Azure B content of at least 2%,
b. dissolving and mixing in water methylene blue in a stainless steel reactor to obtain a solution,
c. optionally, filtering the obtained solution,
d. filling the solution into a suitable pharmaceutical container,
e. sterilising the obtained solution, preferably by terminal sterilisation and preferably reaching at least 110° C. for not less than 10 minutes, more preferably reaching at least 120° C. for not less than 10 minutes.

In another possible combination of the embodiments of the second aspect of the invention, the process is as follows:
1. Procedure of weighing and sampling methylene blue
   1. Transfer the active ingredient methylene blue to a controlled area and weigh the active ingredient to reach a final concentration of 5 mg/mL (weight 5 mg of methylene blue or any of its hydrates for each mL of solution to be manufactured). Protect the active ingredient from light during the whole manufacturing process.
2. Preparation of the solution
   1. Add water for injection (approximately 88% of the batch size volume)
   2. Add slowly the total amount of methylene blue sodium into the vessel under continuous stirring until the total dissolution of the API.
   3. Once the active ingredient is completely dissolved, set (if necessary) the temperature of the solution at 22±3° C.
   4. Make up to the final volume with water for injection
   5. Stir the solution for not less than 20 minutes in order to ensure complete homogenization of the solution.
3. Filtration Stage I
   1. Transfer the solution from the stainless steel compounding vessel to a stainless steel holding vessel through a 0.2 μm pore size filter.
4. Filtration Stage II
   1. Transfer the solution from the stainless steel holding vessel to a stainless steel filling vessel through a 0.2 μm pore size filter.
5. Filling of the vials
   1. Fill aseptically 1 mL, 2 mL or 10 mL of the active solution into 10 mL type I clear glass vials and close them with a stopper and an aluminium seal.
6. Terminal sterilisation
   1. Place the filled vials in the autoclave
   2. Sterilise for 20 minutes between 121° C. and 124° C.
   3. Cool down the sterilised vials to room temperature.
   4. Perform visual inspection of the vials and store them protected from light.

In a third aspect of the invention, an industrial pharmaceutical batch of the composition of the first aspect manufactured by the process of the second aspect is disclosed. Said pharmaceutical batch ranges between 15 L and 120 L of methylene blue aqueous solution.

In a further embodiment, said industrial pharmaceutical batch is packaged in glass vials, ampoules or pre filled syringes.

In another embodiment, each presentation contains 2 mL or 10 mL.

In a preferred embodiment, the pharmaceutical batch yields 13636 vials of the 2 mL presentation.

In a preferred embodiment, the pharmaceutical batch yields 9389 vials of the 10 mL presentation.

Further aspects and embodiments of the present invention can be found in the following numbered embodiments:
1. A sterile aqueous pharmaceutical composition consisting of methylene blue as active ingredient and water, wherein
   a. the Azure B impurity content of said composition ranges from 2.0% to 3.0% with respect to the active ingredient and,
   b. the pH of said composition is below 4.0 and,
   c. the total metal content of said pharmaceutical composition is above 20 ppm, preferably above 50 ppm, more preferably above 75 ppm, even more preferably above 100 ppm.
2. The pharmaceutical composition according to the preceding embodiment, wherein the Azure B impurity content of said composition ranges from 2.1% to 3.0%, preferably from 2.3% to 3.0%.
3. The pharmaceutical composition according to any of the preceding embodiments, wherein the pH is from 3.2 to 3.9, preferably from 3.3 to 3.8.
4. The pharmaceutical composition according to any of the preceding embodiments, wherein the metal content in the composition is above 120 ppm, preferably above 130 ppm, more preferably above 150 ppm.
5. The pharmaceutical composition according to the preceding embodiment, wherein the Iron (Fe) content with respect to the active ingredient is above 20 ppm, preferably above 50 ppm, more preferably above 100 ppm, even more preferably, above 120 ppm.
6. The pharmaceutical composition according to the preceding embodiment, wherein the Iron (Fe) content with respect to the active ingredient is above 100 ppm.
7. The pharmaceutical composition according to embodiment 4, wherein the Aluminium (Al) content with respect to the active ingredient is above 5 ppm, preferably above 8 ppm, more preferably above 10 ppm, even more preferably, above 20 ppm.
8. The pharmaceutical composition according to any of the preceding embodiments, wherein the pharmaceutical composition is packaged in a glass vial, or in a pre-filled-syringe, or in a cartridge or in an ampule, preferably the composition is packaged in a glass vial and sealed with a rubber stopper.
9. The pharmaceutical composition according to any of the previous embodiments, wherein the composition is to be parenterally administered to a human.
10. The pharmaceutical composition according to any of the preceding embodiments for use as a medicament.
11. The pharmaceutical composition according to any of the preceding embodiments for the use in the treatment of dementia, cognitive impairment, Alzheimer or methemoglobinemia.
12. The pharmaceutical composition according to any of the preceding embodiments having an assay value within 95%-105% and total organic impurity content below 3% after 6 months storage under 40° C./75% RH packaged in glass vials with rubber stopper.
13. The pharmaceutical composition according to any of the preceding embodiments, wherein the methylene blue concentration in the aqueous solution is 5 mg/mL.
14. A process for preparing a pharmaceutical composition according to any of the preceding embodiments comprising the steps of:
   a. weighing the methylene blue,
   b. in a reactor, dissolving and mixing methylene blue of step a. in water to obtain a solution,
   c. optionally, filtering the obtained solution,
   d. filling the solution into a suitable pharmaceutical container,
   e. sterilising the obtained solution
15. A process according to the preceding embodiment, wherein the reactor material is metallic, preferably stainless steel.
16. A process according to any of the two preceding embodiments, wherein the solution of step c) is filtered through a 0.2 μm pore size filter.
17. The process according to the preceding embodiment, wherein the filtration is performed twice.
18. A process according to any of the four preceding embodiments, wherein the suitable pharmaceutical container is a glass vial sealed with a rubber stopper.
19. A process according to any of the five preceding embodiments, wherein the pH of the aqueous solution ranges between 3.2 and 3.9.
20. A process according to any of the six preceding embodiments, wherein at least 15 Liters of sterile aqueous solution are obtained.

### EXAMPLES

### I. Preparation of Methylene Blue Infection USP Compositions at 5 mg/mL

Methylene blue pharmaceutical compositions 1 to 7 for parenteral administration were prepared. General preparation method is described below:
Weight the necessary amount of methylene blue to reach a final concentration of 5 mg/ml
Add water for injection in a stainless steel vessel
Add slowly the total amount of methylene blue into the vessel under continuous stirring until the total dissolution of the methylene blue
Add water for injection until to reach the final volume
Stir the resulting solution to ensure complete homogenization of the solution
Filter twice the solution through a 0.2 μm pore size filter
Fill vials with the obtained solution and seal them with a rubber stopper
Place the filled vials in the autoclave and sterilise them

### II. Analysis of the Prepared Compositions

Compositions 1 to 7 were prepared as described in previous point I.

| | Comp 1 | Comp 2 | Comp 3 | Comp 4 | Comp 5 | Comp 6 | Comp 7 |
|---|---|---|---|---|---|---|---|
| Reactor material | Stainless | Glass | Stainless | Stainless | Stainless | Stainless | Stainless |
| | steel | | steel | steel | steel | steel | steel |
| pH | 4.1 | 4.6 | 4.6 | 3.2 | 3.9 | 3.8 | 3.8 |
| Azure B impurity | 1.9 | 2.4 | 2.5 | 2.3 | 2.4 | 2.6 | 2.5 |
| (%) | | | | | | | |
| Total impurity | 1.9 | 2.4 | 2.5 | 2.3 | 2.4 | 2.5 | 2.5 |
| content (%) | | | | | | | |
| Metal content | 23.63 | 131.0 | 105.2 | 192.8 | 131.2 | 35.4 | 140.1 |
| (ppm) | | | | | | | |
| Iron content | ND | 56 | 52 | 94 | 102 | NC | 112 |
| (ppm) | | | | | | | |
| Aluminium | ND | 8 | 8 | 26 | 24 | 16 | ND |
| content (ppm) | | | | | | | |
| Assay (%) | 94.7 | 96.6 | 95.6 | 100.1 | 100.1 | 99.7 | 96.2 |

ND: Not detected

Compositions 2, 3, 6 and 7 were adjusted for pH to obtain comparative examples. Anhydrous citric acid and sodium citrate dihydrate were used at q.s. to arrive at the pH shown in the table.

Compositions 4 and 5 were not adjusted for pH and consist of methylene blue and water. The metal and impurity content of the active ingredient did not increase during manufacturing.

### III. Stability of the Prepared Compositions

During manufacturing of all the compositions, we did not identify a significant increase in the total organic impurities before and after manufacturing. That is, the organic impurities carried by the active ingredient did not increase during manufacturing nor during a stability period of 6 months stored at 40° C./75% RH (relative humidity) or a period of 24 months stored at 25° C./60% RH.

### IV. General Formula of the Prepared Compositions

| Ingredient | Quantity |
|---|---|
| Methylene blue | 5 mg/mL |
| Water for Injection | q.s. to 1 mL |

Commercial batch size manufactured: 15 Liters
Presentations packaged: 2 mL glass vial and 10 mL glass vial

1. A sterile aqueous pharmaceutical composition, consisting of: methylene blue as active ingredient and water, wherein
a. an Azure B impurity content of said composition ranges from 2.0 wt. % to 3.0 wt. % with respect to the active ingredient,
b. a pH of said composition is below 4.0, and
c. a total metal content of said pharmaceutical composition is above 20 ppm.

2. The pharmaceutical composition according to claim 1, wherein the Azure B impurity content of said composition ranges from 2.1 wt. % to 3.0 wt. %.

3. The pharmaceutical composition according to claim 1, wherein the pH is from 3.2 to 3.9.

4. The pharmaceutical composition according to claim 1, wherein he metal content in the composition is above 120 ppm.

5. The pharmaceutical composition according to claim 1, wherein an Iron (Fe) content with respect to the active ingredient is above 20 ppm.

6. The pharmaceutical composition according to claim 1, wherein an Iron (Fe) content with respect to the active ingredient is above 100 ppm.

7. The pharmaceutical composition according to claim 1, wherein an aluminum (Al) content with respect to the active ingredient is above 5 ppm.

8. The pharmaceutical composition according to claim 1, wherein the pharmaceutical composition is packaged in a glass vial, or in a pre-filled-syringe, or in a cartridge, or in an ampule.

9. The pharmaceutical composition according to claim 1, wherein the composition is suitable to be parenterally administered to a human.

10. A medicament, comprising: the pharmaceutical composition according to claim 1.

11. A method of treating dementia, cognitive impairment, Alzheimer's disease or methemoglobinemia, said method comprising: administering the pharmaceutical composition according to claim 1 to a subject in need thereof.

12. The pharmaceutical composition according to claim 1, having an assay value within 95 wt. %-105 wt. % and a total organic impurity content below 3 wt. % after 6 months storage under 40° C./75% RH (relative humidity) packaged in a glass vial with a rubber stopper.

13. The pharmaceutical composition according to claim 1, wherein said composition is an aqueous solution and a methylene blue concentration in the aqueous solution is 5 mg/mL.

14. A process for preparing a pharmaceutical composition according to claim 1, said process comprising:
a. weighing the methylene blue,
b. in a reactor, dissolving and mixing the methylene blue of step a. in water to obtain an aqueous solution,
c. optionally, filtering the obtained aqueous solution,
d. filling the aqueous solution into a suitable pharmaceutical container,
e. sterilizing the obtained solution, thereby obtaining a sterile aqueous solution of said pharmaceutical composition.

15. The process according to claim 14, wherein the reactor material is metallic.

16. The process according to claim 14, wherein the solution in step c) is filtered through a 0.2 μm pore size filter.

17. The process according to claim 14, wherein a filtration through a 0.2 μm pore size filter is performed twice.

18. The process according to claim 14, wherein the suitable pharmaceutical container is a glass vial sealed with a rubber stopper.

19. The process according to claim 14, wherein a pH of the aqueous solution of step d ranges between 3.2 and 3.9.

20. The process according to claim 14, wherein at least 15 liters of sterile aqueous solution are obtained.
